(19) United States
(12) Patent Application Publication
(10) Pub. No.: US 2019/0160054 A1
(43) Pub. Date: May 30, 2019

(54) PHARMACEUTICAL COMBINATION OF NINTEDANIB, TRIFLURIDINE AND TIPIRACIL FOR TREATING COLORECTAL CANCER

(71) Applicant: Boehringer Ingelheim International GmbH, Ingelheim am Rhein (DE)

(72) Inventors: Soetkin VLASSAK, Neervelp (BE); Yasutoshi KUBOKI, Kashiwa (JP); Akihiro SATO, Kashiwa (JP); Takayuki YOSHINO, Kashiwa (JP)

(21) Appl. No.: 16/091,157

(22) PCT Filed: Apr. 10, 2017
(86) PCT No.: PCT/EP2017/058552

§ 371 Date: Oct. 4, 2018

Related U.S. Application Data
(60) Provisional application No. 62/321,983, filed on Apr. 13, 2016.

(51) Int. Cl. A61K 31/496; A61K 31/513; A61K 9/48

(52) U.S. Cl. CPC A61K 31/496; A61K 31/513; A61K 9/485; A61N 5/10; A61K 9/4866; A61K 9/4875; A61K 9/4833; A61K 9/4858

(57) ABSTRACT

The present invention relates to a pharmaceutical combination which may be useful for the treatment of diseases which involve cell proliferation, especially metastatic colorectal cancer comprising 3-Z-[1-(4-(N-((4-methyl-piperazin-1-yl)-methylcarbonyl)-N-methyl-amino)-anilino)-1-phenyl-methylene]-6-methoxycarbonyl-2-indo lino ne or a pharmaceutically acceptable salt thereof, and 2′-deoxy-5-(trifluoromethyl)uridine or a pharmaceutically acceptable salt thereof, and 5-chloro-6-[(2-iminopyrrolidin-1-yl)methyl]pyrimidine-2,4(1H,3H)-dione or a pharmaceutically acceptable salt thereof, wherein the molar ratio of 2′-deoxy-5-(trifluoromethyl)uridine or a pharmaceutically acceptable salt thereof, and 5-chloro-6-[(2-iminopyrrolidin-1-yl)methyl]pyrimidine-2,4(1H,3H)-dione or a pharmaceutically acceptable salt thereof, is 1:0.5

The present invention relates to a pharmaceutical combination which may be useful for the treatment of diseases which involve cell proliferation, more specifically colorectal cancer. The invention also relates to a method for the treatment of diseases with high unmet medical need, comprising simultaneous, separate or sequential administration of effective amounts of specific active compounds and/or co-treatment with radiation therapy, in a ratio which provides an additive and/or synergistic effect, and to the combined use of these specific compounds and/or radiotherapy for the manufacture of corresponding pharmaceutical combination preparations.

The present invention relates more specifically to a pharmaceutical combination comprising the compound nintedanib, i.e. 3-Z-[1-(4-(N-((4-methyl-piperazin-1-yl)-methylcarbonyl)-N-methyl-amino)-anilino)-1-phenyl-methylene]-6-methoxycarbonyl-2-indolinone (Compound A) or a pharmaceutically acceptable salt thereof and a mixture of compounds (Compound Mixture B) or a pharmaceutically acceptable salt thereof, optionally in combination with radiotherapy.

Compound Mixture B is a mixture of trifuridine, also known as 2′-deoxy-5-(trifluoromethyl)uridine, TFT (CAS 733030-01-8)

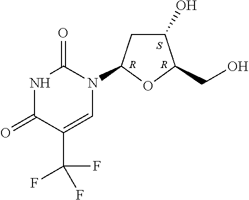

and tipriracil, i.e. 5-chloro-6-[(2-iminopyrrolidin-1-yl)-methyl]pyrimidine-2,4(1H,3H)-dione (CAS 183204-74-2):

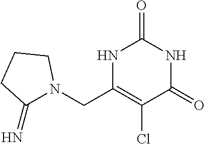 [SMILES: N=C1CCCN1Cc1[nH]c(=O)[nH]c(=O)c1Cl]

in the molar ration of 1:0.5.

### BACKGROUND TO THE INVENTION

The compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone (Compound A) is an innovative compound having valuable pharmacological properties, especially for the treatment of oncological diseases, immunologic diseases or pathological conditions involving an immunologic component, or fibrotic diseases.

The chemical structure of this compound is depicted below as Formula A.

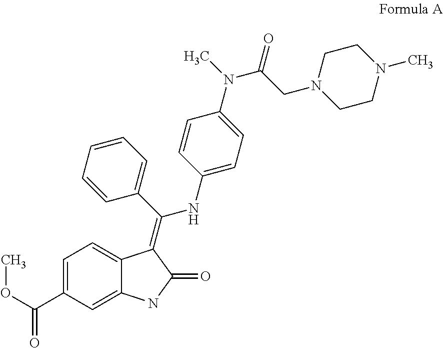 [SMILES: COC(=O)c1ccc2c(c1)NC(=O)/C2=C(\Nc1ccc(N(C)C(=O)CN2CCN(C)CC2)cc1)c1ccccc1]

The base form of this compound is described in WO 01/27081, the monoethanesulphonate salt form is described in WO 2004/013099 and various further salt forms are presented in WO 2007/141283. The use of this molecule for the treatment of immunologic diseases or pathological conditions involving an immunologic component is being described in WO 2004/017948, the use for the treatment of oncological diseases is being described in WO 2004/096224 and the use for the treatment of fibrotic diseases is being described in WO 2006/067165.

The monoethanesulphonate salt form of this compound presents properties which makes this salt form especially suitable for development as medicament. The chemical structure of 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate is depicted below as Formula A1.

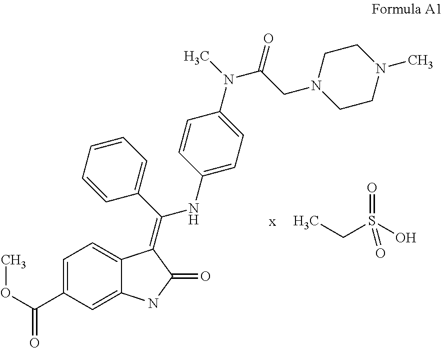

Preclinical studies have shown that this compound is a highly potent, orally bioavailable inhibitor of vascular endothelial growth factor receptors (VEGFRs), platelet-derived growth factor receptors (PDGFRs) and fibroblast growth factor receptors (FGFRs) that suppresses tumor growth through mechanisms inhibiting tumor neovascularization. It has further been shown that this compound inhibits signaling in endothelial- and smooth muscle cells and pericytes, and reduces tumor vessel density.

Furthermore, this compound shows in vivo anti-tumor efficacy in all models tested so far at well tolerated doses. The following table shows the results of the in vivo anti-tumor efficacy testing in xenograft models and in a syngeneic rat tumor model.

| Cancer | Model | Efficacy |
|---|---|---|
| Colorectal | HT-29 | T/C 16% @ 100 mg/kg/d |
| | HT-29 large | tumor volume reduction |
| | tumors | |
| Glioblastoma | GS-9L syngeneic | T/C 32% @ 50 mg/kg/d |
| | rat | |
| Head and neck | FaDu | T/C 11% @ 100 mg/kg/d |
| Lung (non-small-cell) | NCI-H460 | T/C 54% @ 25 mg/kg/d |
| | Calu-6 | T/C 24% @ 50 mg/kg/d |
| Ovarian | SKOV3 | T/C 19% @ 50 mg/kg/d |
| Prostate (hormone- | PAC-120 | T/C 34% @ 100 mg/kg/d |
| dependent) | | |
| Renal | Caki-1 | T/C 13% @ 100 mg/kg/d |
| Pancreas (murine | Rip-Tag | interference with tumor |
| transgenic) | | formation |

T/C represents the reduction of tumor size in % of the control

This compound is thus suitable for the treatment of diseases in which angiogenesis or the proliferation of cells is involved. It has been approved for idiopatic pulmonary fibrosis, e.g. in the US, Japan and for the EU. In Europe it has been also approved for Non-Small-Cell Lung Cancer/Carcinoma (NSCLC). A global phase III study, called as the LUME-Colon 1 study, comparing nintedanib monotherapy versus placebo in pts with mCRC refractory to standard therapies is ongoing (NCT02149108).

Compound Mixture B is a mixture of TFT, i.e. 2′-deoxy-5-(trifluoromethyl)uridine, (also known as trifluridine) and tipiracil, i.e. 5-chloro-6-[(2-iminopyrrolidin-1-yl)-methyl]pyrimidine-2,4(1H,3H)-dione (CAS 183204-74-2) in the molar ration of 1:0.5.

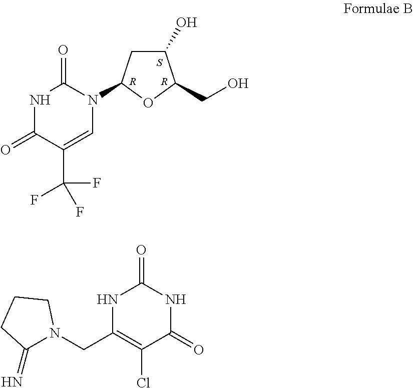

More specifically compound mixture B1 is a mixture of TFT and tipiracil hydrochloride (i.e. 5-chloro-6-[(2-iminopyrrolidin-1-yl)methyl]pyrimidine-2,4(1H,3H)-dione monohydrochloride) in the ratio 1:0.5.

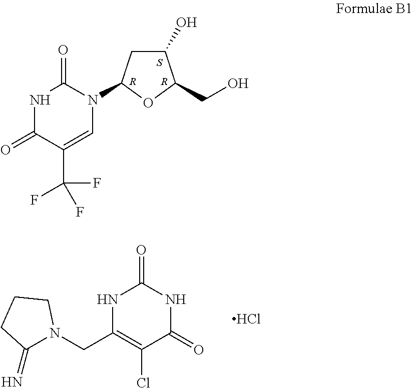

See: Future Oncol. (2016) 12(2), 153-163 and Expert Review of Clinical Pharmacology (2016), Vol. 9, No. 3, 355-365.

Compound mixture B (to be more accurate: Compound mixture B1) has been approved in the US under the tradename Lonsurf® (Taiho Pharmaceutical Co., Ltd) and the lab code TAS-102 for the treatment of patients with metastatic colorectal cancer who have been previously treated with fluoropyrimidine-, oxaliplatin-, and irinotecan-based chemotherapy, an anti-VEGF biologic product, and an anti-EGFR monoclonal antibody, if RAS wild-type.

The approval was based on the demonstration of improved overall survival (OS) in a multicenter, double-blind, placebo-controlled phase III trial (TPU-TAS-102-301). A total of 800 patients with previously treated metastatic colorectal cancer were randomly allocated (2:1) to receive trifluridine/tipiracil (N=534) plus best supportive care (BSC) or matching placebo (N=266) plus BSC. Key eligibility criteria included ECOG 0-1, absence of brain metastasis, and absence of ascites requiring drainage in the past four weeks. Patients received 35 mg/m² trifluridine/tipiracil (based on trifluridine component) or matching placebo orally twice daily on days 1-5 and 8-12 of each 28-day cycle until disease progression or unacceptable toxicity.

A statistically significant improvement in OS was demonstrated [HR 0.68 (95% CI: 0.58, 0.81), p<0.001, stratified log-rank test]; the median OS was 7.1 and 5.3 months in the trifluridine/tipiracil and placebo arms, respectively. PFS was also improved in patients randomly allocated to receive trifluridine/tipiracil [HR 0.47 (95% CI: 0.40, 0.55), p<0.001, stratified log-rank test].

The recommended dose and schedule for trifluridine/tipiracil is 35 mg/m² (based on trifluridine component) orally twice daily within one hour of completion of morning and evening meals on days 1 through 5 and days 8 through 12 of each 28-day cycle until disease progression or unacceptable toxicity.

### SUMMARY OF THE INVENTION

One embodiment of the invention is a pharmaceutical combination comprising components
(i) 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, and
(ii) 2′-deoxy-5-(trifluoromethyDuridine or a pharmaceutically acceptable salt thereof, and
(iii) 5-chloro-6-[(2-iminopyrrolidin-1-yl)methyl]pyrimidine-2,4(1H,3H)-dione or a pharmaceutically acceptable salt thereof,

wherein the molar ratio of component (ii), 2′-deoxy-5-(trifluoromethyl)uridine or a pharmaceutically acceptable salt thereof, and component (iii), 5-chloro-6-[(2-iminopyrrolidin-1-yl)methyl]pyrimidine-2,4(1H,3H)-dione or a pharmaceutically acceptable salt thereof, is 1:0.5.

A further preferred embodiment of the invention is the pharmaceutical combination of the preceding paragraph, in which the pharmaceutically acceptable salt of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone is its monoethanesulphonate salt form.

A further preferred embodiment of the invention is the pharmaceutical combination of one of the preceding paragraphs, in which the pharmaceutically acceptable salt of the compound 5-chloro-6-[(2-iminopyrrolidin-1-yl)methyl]pyrimidine-2,4(1H,3H)-dione is the hydrochloride form.

Preferred is the pharmaceutical combination according to one of the preceding paragraphs, wherein components (ii) and (iii) form a mixture for simultaneous use of both components. Even more preferred is the pharmaceutical combination according to one of the preceding paragraphs, wherein component (i) and the mixture of this paragraph is for sequential use.

A further preferred embodiment of the invention is the pharmaceutical combination of one of the preceding paragraphs which is further adapted for a co-treatment with radiotherapy.

Preferred is the pharmaceutical combination according to one of the preceding paragraphs for use in the treatment of colorectal cancer, more preferred metastatic colorectal cancer, even more preferred metastatic colorectal cancer refractory or intolerant to standard therapies. The standard therapies are preferably selected from the group consisting of fluoropyrimidine, irinotecan, oxaliplatin, anti-angiogenesis inhibitor and anti-EGFR antibody (if wild-type RAS).

A further preferred embodiment of the invention is the pharmaceutical combination of one of the preceding paragraphs, wherein component (i) is intended to be administered in the amount of 100 mg of the free base, twice daily, more preferred 150 mg of the free base, twice daily, most preferred 200 mg of the free base, twice daily.

Most preferred is an embodiment of the invention is the pharmaceutical combination of one of the preceding paragraphs, wherein component (ii) is intended to be administered in the amount of 35 mg of the free base per m², twice daily at days 1 to 5 and days 8 to 12 of a treatment cycle of 28 days and wherein component (iii) is to be administered simultaneously with component (ii) according to the molar ratio (ii):(iii) of 1:0.5. It is highly preferred that component (ii) and (iii) form a mixture for simultaneous use of both components. Even more preferred is the pharmaceutical combination, wherein component (i) and the mixture of this paragraph is for sequential use.

A further embodiment of the invention are compounds
(iv) 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, and
(v) 2′-deoxy-5-(trifluoromethyfluridine or a pharmaceutically acceptable salt thereof, and
(vi) 5-chloro-6-[(2-iminopyrrolidin-1-yl)methyl]pyrimidine-2,4(1H,3H)-dione or a pharmaceutically acceptable salt thereof,
for combined use in the treatment of colorectal cancer, wherein the molar ratio of compound (v), 2′-deoxy-5-(trifluoromethyl)uridine or a pharmaceutically acceptable salt thereof and compound (iv), 5-chloro-6-[(2-iminopyrrolidin-1-yl)methyl]pyrimidine-2,4(1H,3H)-dione or a pharmaceutically acceptable salt thereof, is 1:0.5.

A further embodiment of the invention is a method for treating metastatic colorectal cancer comprising administering to a patient in need thereof

compound (viii): 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, in combination with

compound (ix): 2′-deoxy-5-(trifluoromethyl)uridine or a pharmaceutically acceptable salt thereof, and

compound (x): 5-chloro-6-[(2-iminopyrrolidin-1-yl)methyl]pyrimidine-2,4(1H,3H)-dione or a pharmaceutically acceptable salt thereof, wherein the molar ratio of

compound (ix) and compound (x) is 1:0.5.

A further embodiment of the invention is a method for treating metastatic colorectal cancer comprising administering to a patient in need thereof

a pharmaceutical combination consisting essentially of compound (viii), compound (ix) and compound (x) wherein the molar ratio of compound (ix) and compound (x) is 1:0.5.

A further embodiment of the invention is a method for treating metastatic colorectal cancer comprising administering to a patient in need thereof

a pharmaceutical combination consisting of compound (viii), compound (iv) and compound (x) wherein the molar ratio of compound (ix) and compound (x) is 1:0.5.

A further embodiment of the invention is a method for treating metastatic colorectal cancer comprising
identifying a patient in need of treatment of colorectal cancer
treating the patient with a combination therapy comprising compound (viii), compound (iv) and compound (x) wherein the molar ratio of compound (ix) and compound (x) is 1:0.5.

### LEGEND TO THE FIGURES

FIG. 1 shows the current clinical course of all patients in the first phase of the study. Blue, red and green bares stand for the administration of 200 mg, 150 mg, and 100 mg nintedanib. PD means tumor progression, PR means partial response, SD means stable disease, DILI means liver enzyme elevation, + means death.

### DETAILED DESCRIPTION OF THE INVENTION

As already mentioned hereinbefore, the present invention relates to a pharmaceutical combination comprising an effective amount of the Compound A or a pharmaceutically acceptable salt thereof and an effective amount of the Compound mixture B or a pharmaceutically acceptable salt thereof.

A combination treatment of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic or targeted agent in addition to a combination treatment of the invention. Surgery may comprise the step of partial or complete tumor resection, prior to, during or after the administration of the combination treatment as described herein.

According to another aspect of the present invention, the effect of a method of treatment of the present invention is expected to be at least equivalent to the addition of the effects of each of the components of said treatment used alone, that is, of each of the compounds and ionising radiation used alone.

According to another aspect of the present invention the effect of a method of treatment of the present invention is expected to be greater than the addition of the effects of each of the components of said treatment used alone, that is, of each of the compounds and ionising radiation used alone.

According to another aspect of the present invention the effect of a method of treatment of the present invention is expected to be a synergistic effect. A combination treatment is defined as affording a synergistic effect if the effect is therapeutically superior, as measured by, for example, the extent of the response, the duration of response, the response rate, the stabilization rate, the duration of stabilization, the time to disease progression, the progression free survival or the overall survival, to that achievable on dosing one or other of the components of the combination treatment at its conventional dose. For example, the effect of the combination treatment is synergistic if the effect is therapeutically superior to the effect achievable with one component alone. Further, the effect of the combination treatment is synergistic if a beneficial effect is obtained in a group of patients that does not respond (or responds poorly) to one component alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the duration of response, the response rate, the stabilization rate, the duration of stabilization, the time to disease progression, the progression free survival or the overall survival, is equivalent to that achievable on dosing conventional amounts of the components of the combination treatment.

In particular, synergy is deemed to be present if the conventional dose of one of the components may be reduced without detriment to one or more of the extent of the response, the duration of response, the response rate, the stabilization rate, the duration of stabilization, the time to disease progression, the progression free survival or the overall survival, in particular without detriment to the duration of the response, but with fewer and/or less troublesome side-effects than those that occur when conventional doses of each component are used.

The advantages of the present invention are the potential for an improved clinical benefit for cancer patients treated with this pharmaceutical combination involving one or more of the following mechanisms:

Additive or synergistic antitumor effect mediated by the combination of two different anticancer principles and target structures: Compound A1 is an antiangiogenic compound targeting the tumor vasculature (endothelial cells, pericytes, and smooth muscle cells) with suppression of tumor (re-)growth and metastatic spread; Compound mixture B1 is a cyctotoxic mixture interacting with de novo DNA synthesis pathways. Unlike normal cells, cancer cells are genetically instable, causing them to replicate inaccurately. As tumors progress, this genetic instability leads to subpopulations of tumor cells with different biological features. An antitumor treatment like Compound mixture B1 may terminate even the majority of tumor tissue, however, finally, some cell clones will become refractory. After the treatment-sensitive cells have been killed, the resistant cells may rapidly divide again to restore a tumor that is inherently resistant to the therapy. Therefore, simultaneous targeting of different principles driving cancer growth and spread with the described combination of Compound A1 and Compound Mixture B1 reduce the risk of primary and secondary tumor resistance and tumor escape as well. The validity of such approaches has been demonstrated for combination and multimodality treatment in a variety of solid and hematologic human malignancies, but not for the combination object of the present invention, i.e. the combination of Compound A1 and Compound Mixture B1. Of importance in the context of the present invention may be the fact that Compound A1 primarily acts on the genetically stable cells of the tumor vasculature which are less prone to spontaneous mutation and resistance development as compared to the malignant cells. Moreover, one component of the Compound Mixture B1, tipiracil HCl not only has the function of improving the half-life of TFT by reducing its degradation by thymidine phosphorylase (TP, thymidine:phosphate deoxy-alpha-D-ribosyltransferase) but has also antiangiogenic properties by inhibiting TP. TP is identical to platelet derived endothelial call growth factor (PD-ECGF) and can promote growth in vivo by mechanism that include endothelial cell migration and angiogenesis. TP also protects cells form hypoxia-induced apoptosis, see Future Oncol. (2016) 12(2), 153-163 and Expert Review of Clinical Pharmacology (2016), Vol. 9, No. 3, 355-365 pages 156-157 and 357, respectively.

Phase I/II Study Results

A phase I/II study to assess efficacy and safety for the combination of Compound A1 with Compound Mixture B1 was initiated.

Methods:

The key eligibility criteria were patients with metastatic colorectal cancer refractory or intolerant to fluoropyrimidine, irinotecan, oxaliplatin, anti-angiogenesis inhibitor and anti-EGFR antibody (if wild-type RAS) and without prior regorafenib and Compound Mixture B1 treatment; at least one measurable lesion; and ECOG performance status of 0 or 1. Phase I part was designed to determine the recommended phase II dose (RP2D) in a “3+3” cohort-based dose escalation design of Compound A1 (150 mg BID every day on level 1 and 200 mg BID every day on level 2) with a fixed dose of Compound Mixture B1 (35 mg/m² based on the amount of TFT BID on days 1-5 and 8-12 q4w).

(1 cycle is 28 days; Compound A1 (150 mg or 200 mg) is orally administered twice daily and Compound Mixture B1 (35 mg/m²) is orally administered twice daily in 1-5 days and 8-12 days and two weeks off).

Primary endpoint of the phase II part was an investigator-assessed progression free survival (PFS) rate at 16 weeks in pts receiving the combination with RP2D. Using a single stage binomial design, this study required 54 patients, with an investigator-assessed PFS rate at 16 weeks of 40% deemed promising and 25% unacceptable (alpha=0.1; beta=0.2). Secondary endpoints included overall survival (OS), PFS, objective response rate, disease control rate, safety, and pharmacokinetic parameters. The enrollment to phase II part began in January 2016. Clinical trial information: UMIN000017114.

Key Inclusion Criteria:
1) Written informed consent
2) >=20 years old
3) Histologically or cytologically proven adenocarcinoma of colon or rectum (except appendiceal cancer), and KRAS test were carried out.
4) Prior chemotherapy was discontinued due to disease progression, or an adverse event.
5) No prior treatment with Regorafenib
6) ECOG Performance Status of 0 or 1
7) Adequate oral intake
8) With measurable disease according to RECIST
9) Adequate organ function within 14 days prior to enrollment.
   a. Hemoglobin>=9.0 g/d
   b. Absolute neutrophil count>=1,500/mm3
   c. Platelet count>=100,000/mm3
   d with no liver metastasis, AST/ALT<=ULNx1.5 and T-Bil<=UNL with liver metastasis, AST/ALT<=ULNx2.5 and T-Bil<=UNL
   e Serum creatinine<=1.5 mg/dL
10) Women of childbearing potential must have a negative pregnancy test within 7 days prior to enrollment. Patients who do have intention that uses effective contraception method until 6 months after administering investigational drug.
11) Patient is willing or able to comply with the protocol.

Key Exclusion Criteria:
1) Serious coexisting illness as follows.
   a. Active double cancer(s)
   b. CNS metastasis
   c. Uncontrollable infectious disease
   d. Uncontrollable pleural effusion, ascites, or cardiac effusion
   e. Ileus, interstitial pneumonia, renal failure, hepatic failure or cerebrovascular disorder
   f. Uncontrolled diabetic
   g. Uncontrolled hypertension
   h. Cardiac infarction, serious angina or New York Heart Association Class III or IV within 6 months prior to the registration
   i Gastrointestinal bleeding
   j Positive HBs antigen, HCV antibody or HIV antibody
   k. Need immunosuppressive therapy
   l. Uncontrolled mental disease or the psychotic manifestation
2) Receive the treatment as follows.
   a) Major surgery therapy within 4 weeks prior to enrollment.
   b) Chemotherapy within 3 weeks prior to enrollment.
   c) Wide field radiotherapy 4 weeks prior to enrollment or local radiotherapy 2 weeks prior to enrollment.
   d) Other investigational drugs within 4 weeks prior to enrollment.
3) Prior treatment with TAS-102 or Nintedanib
4) Unrecovered AEs related to prior treatment are Grade>=2 according to CTCAE.
5) Blood transfusion or G-CSF within 14 days prior to enrollment.
6) Serious renal failure or proteinuria>=2+ within 7 days prior to enrollment.
7) Thromboembolic event or serious Pulmonary complaints within one year
8) Uncomplete cure wound or traumatic fracture
9) Tendency to hemorrhages or taking antithrombotic medication
10) Women during pregnancy or lactation,
11) Patient is judged by the investigator to be inappropriate for study participation for any reason.

Results

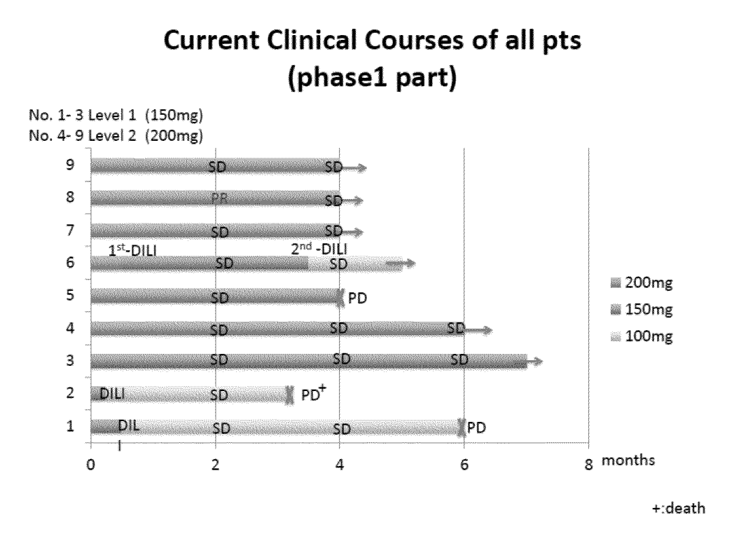

See FIG. 1. Nine patients were examined in the first phase. The administration of 150 mg nintedanib was planned for patients 1-3 and 200 mg for patients 4-9. The dose was adjusted in patients 1, 2 and 6: once during the first two month for patients 1 and 2 to 100 mg nintedanib and twice for patient 6 first form 200 mg to 150 mg during the first two months and form 150 mg to 100 mg during month 2 to 4 after enrolment of the patient in the trial. Patients 1, 2 and 5 progressed after 2 to 6 months and died. However, 6 form nine patients displayed at least a stable disease for at least 4 months and patient 8 indeed showed a partial response.

TABLE 1
Efficacy Evaluation (Phase I part)

| | | | | Image | Image | | |
|---|---|---|---|---|---|---|---|
| | | | | assessment | assessment | Best | RAS |
| Site | SSID | Status | Starting Date | (8 w) | (16 w) | response | status |
| NCCE | 0001 | Off (24 wPD) | 31 AUG. 2015 | SD | SD | SD | MT |
| | | | | (−5.0%) | (+18.6%) | | |
| JFCR | 0002 | Death | 3 SEP. 2015 | SD | Terminated | SD | WT |
| | | (27 JAN. 2016) | | (+2.0%) | by NTL PD | | |
| | | | | | 3 Cy | | |
| | | | | | completed | | |
| | | | | | (TL: −1.0%) | | |
| NCCE | 0003 | On-going | 3 SEP. 2015 | SD | SD | SD | MT |
| | | | | (−3.0%) | (−4.9%) | | |
| NCCE | 0004 | On-going | 8 OCT. 2015 | SD | SD | SD | MT |
| | | | | (−0.0%) | | | |
| Shikoku | 0005 | Off (16 wPD) | 15 OCT. 2015 | SD | PD | SD | WT |
| | | | | (+1.7%) | | | |
| Shikoku | 0006 | On-going | 22 OCT. 2015 | SD | SD | SD | MT |
| | | | | (−20.6%) | (−28%) | | |
| JFGR | 0007 | On-going | 26 NOV. 2015 | SD | SD | SD | MT |
| | | | | (−5.1%) | (+6%) | | |
| NCCE | 0008 | On-going | 26 NOV. 2015 | PR | SD | PR | MT |
| | | | | (−30.5%) | (−25.0%) | | |
| NCCE | 0009 | On-going | 27 NOV. 2015 | SD | SD | SD | WT |
| | | | | (−10.0%) | (−10.0%) | | |

SD: stable disease,
PR: tumor recession,
PD: tumor progression

TABLE 2
Patients Characteristics (Phase II part)

| | Phase 2 part |
|---|---|
| | N = 26 |
| Gender | |
| male | 18 |
| female | 8 |
| Age (years) | Median 64.5 |
| | (44-77) |
| Performance Status | |
| 0 | 21 |
| 1 | 5 |
| Diagnosis | |
| Colon | 16 |
| Rectum | 10 |
| (K)RAS status | |
| wild | 10 |
| mutant | 16 |
| BSA | |
| >1.7 | 13 |
| 1.5-1.7 | 9 |
| <1.5 | 4 |

TABLE 3
Efficacy Evaluation (phase 2 part)

| | | Image | Image | |
|---|---|---|---|---|
| | | assessment | assessment | Best |
| SSID | Status | (8 w) | (16 w) | response |
| 0010 | On-going | SD | Not yet | SD |
| | | (−25.0%) | | |
| 0011* | On-going | SD | Not yet | SD |
| | | (+2.3%) | | |
| 0012 | PD | PD | Not yet | PD |
| (DILI)** | | | | |
| 0013 | On-going | SD | Not yet | SD |
| | | (−4.8%) | | |
| 0014 | PD | PD | Not yet | PD |
| 0015 | On-going | SD | Not yet | SD |
| | | (+6.6%) | | |
| 0016 | PD | PD | Not yet | PD |
| 0017 | On-going | SD | Not yet | SD |
| | | (+5.3%) | | |
| 0018* | On-going | Not yet | Not yet | |
| 0019 | On-going | Not yet | Not yet | |
| 0020 | On-going | Not yet | Not yet | |
| 0021 | On-going | Not yet | Not yet | |
| 0022 | On-going | Not yet | Not yet | |
| 0023 | On-going | Not yet | Not yet | |
| 0024 | On-going | Not yet | Not yet | |
| 0025 | On-going | Not yet | Not yet | |
| 0026 | On-going | Not yet | Not yet | |
| 0027 | On-going | Not yet | Not yet | |
| 0028 | PD | — | — | PD |
| | (C1D36) | | | (brain meta. |
| | | | | required |
| | | | | treatment) |
| 0029 | On-going | Not yet | Not yet | |
| 0030 | On-going | Not yet | Not yet | |
| 0031* | On-going | Not yet | Not yet | |
| 0032 | On-going | Not yet | Not yet | |
| 0033 | On-going | Not yet | Not yet | |
| 0034 | On-going | Not yet | Not yet | |
| 0035* | On-going | Not yet | Not yet | |

*BSA < 1.5,
**DILI (BSA 1.52)

Five patients displayed a stable disease whereas 4 progressed before 16 weeks despite treatment.

Five patients form all enrolled 35 patients required at least one dose reduction of nintedanib:

| All enrolled patients, N = 35 | Reason |
|---|---|
| One dose reduction | DILI (cycle 1), N = 3 |
| (N = 4) | Diarrhea grade 2* (cycle 1) N = 1 |
| Two dose reduction | DILI (cycle 1, cycle 4) |
| (N = 1) | |

*continuously for more than 8 days despite supportive therapy, DILI: drug induced liver injury

Three patients required one dose reduction of TAS-102

| All enrolled patients, N = 35 | Reason |
|---|---|
| One dose reduction | Thrombocytopenia grade 4 (cycle 4) N = 1 |
| (N = 3) | Anorexia grade 2* (cycle 4) N = 1 |
| | Fatigue grade 2* (cycle 4) N = 1 |

*dose-reduction was considered necessary takin into account safety at investigators judgment

Liver enzyme elevation occurred during the first treatment course in all but one patient. The second DILI occurred after oral medication with ursodeoxycholic acid was interrupted because liver enzymes were kept within the normal limit for several months (patient No. 6 in phase one part). In the phase two part, liver enzyme elevation occurred in only one patient. These liver enzyme elevations were fully reversible and recovered 1-2 weeks after treatment interruption. The elevations did not exhibit an increase of bilirubin. Serious adverse events occurred in two patients. The causal relationship with treatment was denied in one case. In another case, there was a causal relationship with TAS-102 (anorexia). Accordingly, the continuation of enrolment of more than 20 patients was fine at the dose of nintedanib (200 mg bid) and with TAS-102 35 mg/m², bid, d1-5, d8-12).

TAS-102 Alone:

The phase III double-blind RECOURSE study randomized 800 patients with refractory mCRC in a 2:1 ratio to receive best supportive care plus TAS-102 (n=534) or placebo (n=266). The median age of patients was 63 years and the majority (60%-63%) received ≥4 prior lines of therapy. All patients had received prior fluoropyrimidine, irinotecan, oxaliplatin, and bevacizumab, and 52% had received an EGFR inhibitor. Approximately 20% of patients had received prior treatment with regorafenib.

TAS-102 was administered at 35 mg/m² twice daily with meals for 5 days, with 2 days of rest for 2 weeks followed by a 14-day rest period. The protocol allowed a maximum of 3 dose reductions of 5 mg/m² each. The primary endpoint of the study was OS, with secondary endpoints focused on PFS, overall response rate (ORR), and disease control rate (DCR).

In the RECOURSE study, the median overall survival (OS) for patients with mCRC who received TAS-102 was 7.1 months compared with 5.3 months with placebo (HR, 0.68; P<0.0001). The median progression-free survival (PFS) in the TAS-102 arm was 2 months versus 1.7 months with placebo (HR, 0.48; P<0.0001).

The ORR was 1.6% with TAS-102, which consisted of a complete response in 1 patient and partial responses. The ORR with placebo was 0.4% (P=0.29). Stable disease at 6 weeks was achieved in 42.4% of patients treated with TAS-102. The DCR (partial response, complete response, and stable disease) was 44% with TAS-102 versus 16% with placebo (P<0.001). (See more at: http://www.onclive.com/web-exclusives/fda-approves-tas-102-for-advanced-colorectal-cancer#sthash.YcFYkV5S.dpuf).

The Present Study:

It has to be emphasized that 6/9 patients out of the phase I part, i.e. more than 60% displayed a stable disease or even showed tumor regression up to four months Thus, the combination shows improved efficacy compared to either nintedanib or TAS-102 alone. This is efficacy is quite remarkable given the fact that the patients were refractory or intolerant to other treatments (fluoropyrimidine, irinotecan, oxaliplatin, anti-angiogenesis inhibitor and anti-EGFR antibody).

### Further Embodiments

Further pharmaceutically acceptable salts of the compounds of the combination in accordance with the present invention than those already described hereinbefore may, for example, include acid addition salts. Such acid addition salts include, for example, salts with inorganic or organic acids affording pharmaceutically acceptable anions such as with hydrogen halides or with sulphuric or phosphoric acid, or with trifluoroacetic, citric or maleic acid. In addition, pharmaceutically acceptable salts may be formed with an inorganic or organic base which affords a pharmaceutically acceptable cation. Such salts with inorganic or organic bases include for example an alkali metal salt, such as a sodium or potassium salt and an alkaline earth metal salt such as a calcium or magnesium salt.

In accordance with the present invention, the compounds of the combination may be formulated using one or more pharmaceutically acceptable excipients or carriers, as suitable. Suitable formulations for both compounds A1 and B1 which may be used within the scope of the present invention have already been described in the literature and in patent applications related to these compounds. These formulations are incorporated herein by reference.

In a further preferred embodiment in accordance with the present invention, the formulation for the compound of formula A1 is a lipid suspension of the active substance comprising preferably a lipid carrier, a thickener and a glidant/solubilizing agent, most preferably in which the lipid carrier is selected from corn oil glycerides, diethylenglycolmonoethylether, ethanol, glycerol, glycofurol, macrogolglycerolcaprylocaprate, macrogolglycerollinoleate, medium chain partial glycerides, medium chain triglycerides, polyethylene glycol 300, polyethylene glycol 400, polyethylene glycol 600, polyoxyl castor oil, polyoxyl hydrogenated castor oil, propylene glycol monocaprylate, propylene glycol monolaurate, refined soybean oil, triacetin, triethyl citrate, or mixtures thereof, the thickener is selected from oleogel forming excipients, such as Colloidal Silica or Bentonit, or lipophilic or amphiphilic excipients of high viscosity, such as polyoxyl hydrogenated castor oil, hydrogenated vegetable oil macrogolglycerol-hydroxystearates, macrogolglycerol-ricinoleate or hard fats, and the glidant/solubilizing agent is selected from lecithin, optionally further comprising one or more macrogolglycerols, preferably selected from macrogolglycerol-hydroxystearate or macrogolglycerol-ricinoleate. The lipid suspension formulation may be prepared by conventional methods of producing formulations known from the literature, i.e. by mixing the ingredients at a pre-determined temperature in a pre-determined order in order to obtain a homogenized suspension.

The above formulation may be preferably incorporated in a pharmaceutical capsule, preferably a soft gelatin capsule, characterised in that the capsule shell comprises e.g. glycerol as plasticizing agent, or a hard gelatin or hydroxypropylmethylcellulose (HPMC) capsule, optionally with a sealing or banding. The capsule pharmaceutical dosage form may be prepared by conventional methods of producing capsules known from the literature. The soft gelatin capsule may be prepared by conventional methods of producing soft gelatin capsules known from the literature, such as for example the “rotary die procedure”, described for example in Swarbrick, Boylann, Encyclopedia of pharmaceutical technology, Marcel Dekker, 1990, Vol. 2, pp 269 ff or in Lachmann et al., “The Theory and Practice of Industrial Pharmacy”, 2nd Edition, pages 404-419, 1976, or other procedures, such as those described for example in Jimerson R. F. et al., “Soft gelatin capsule update”, Drug Dev. Ind. Pharm., Vol. 12, No. 8-9, pp. 1133-44, 1986.

The above defined formulation or the above defined capsule may be used in a dosage range of from 0.1 mg to 20 mg of active substance/kg body weight, preferably 0.5 mg to 4 mg active substance/kg body weight. The amount of active substance is calculated as free base.

The above defined capsules may be packaged in a suitable glass container or flexible plastic container, or in an aluminium pouch or double poly bag.

The following examples of carrier systems (formulations), soft gelatin capsules, bulk packaging materials, and of a manufacturing process are illustrative of the present invention and shall in no way be construed as a limitation of its scope.

### Examples of Carrier Systems (Formulations), Soft Gelatin Capsules, Bulk Packaging Materials, and of a Manufacturing Process for the Preparation of a Lipid Suspension Formulation of Compound A1

The active substance in all the Examples 1 to 10 is 3-Z-[1-(4-(N-((4-methyl-piperazin-1-yl)-methylcarbonyl)-N-methyl-amino)-anilino)-1-phenyl-methylene]-6-methoxycarbonyl-2-indolinone-monoethanesulphonate (compound A1).

### Example 1

Lipid Based Carrier System

| | Formulation | | |
|---|---|---|---|
| | A | B | C |
| Ingredients | [%] | [%] | [%] |
| Active Substance | 43.48 | 43.48 | 43.48 |
| Triglycerides, | 28.70 | 37.83 | 38.045 |
| Medium-Chain | | | |
| Hard fat | 27.39 | 18.26 | 18.26 |
| Lecithin | 0.43 | 0.43 | 0.215 |
| Total (Fillmix) | 100.00 | 100.00 | 100.00 |

### Example 2

Lipid Based Carrier System with Additional Surfactant

| Ingredients | [%] |
|---|---|
| Active Substance | 42.19 |
| Triglycerides, | 41.77 |
| Medium-Chain | |
| Hard fat | 12.66 |
| Cremophor RH40 | 2.95 |
| Lecithin | 0.42 |
| Total (Fillmix) | 100.00 |

### Example 3

Hydrophilic Carrier System

| Ingredients | [%] |
|---|---|
| Active Substance | 31.75 |
| Glycerol 85% | 3.17 |
| Purified Water | 4.76 |
| Macrogol 600 | 58.10 |
| Macrogol 4000 | 2.22 |
| Total (Fillmix) | 100.00 |

### Example 4

Soft Gelatin Capsule Containing 50 mg of Active Substance (Free Base)

| | | Formulation | | Formulation |
|---|---|---|---|---|
| | | A | Formulation B | C |
| | | mg per | mg per | mg per |
| Ingredients | Function | capsule | capsule | capsule |
| Active | Active | 60.20 | 60.20 | 60.20 |
| Substance | Ingredient | | | |
| Triglycerides, | Carrier | 40.95 | 53.70 | 54.00 |
| Medium-chain | | | | |
| Hard fat | Thickener | 38.25 | 25.50 | 25.50 |
| Lecithin | Wetting | 0.60 | 0.60 | 0.30 |
| | agent/ | | | |
| | Glidant | | | |
| Gelatin | Film- | 72.25 | 72.25 | 72.25 |
| | former | | | |
| Glycerol 85% | Plasticizer | 32.24 | 32.24 | 32.24 |
| Titanium | Colorant | 0.20 | 0.20 | 0.20 |
| dioxide | | | | |
| Iron oxide A | Colorant | 0.32 | 0.32 | 0.32 |
| Iron oxide B | Colorant | 0.32 | 0.32 | 0.32 |
| Total Capsule | | 245.33 | 245.33 | 245.33 |
| Weight | | | | |

### Example 5

Soft Gelatin Capsule Containing 100 mg of Active Substance

| | | Formulation | | Formulation |
|---|---|---|---|---|
| | | A | Formulation B | C |
| | | mg per | mg per | mg per |
| Ingredients | Function | capsule | capsule | capsule |
| Active | Active | 120.40 | 120.40 | 120.40 |
| Substance | Ingredient | | | |
| Triglycerides, | Carrier | 81.90 | 107.40 | 106.8 |
| Medium-chain | | | | |
| Hard fat | Thickener | 76.50 | 51.00 | 51.00 |
| Lecithin | Wetting | 1.20 | 1.20 | 1.80 |
| | agent/ | | | |
| | Glidant | | | |
| Gelatin | Film- | 111.58 | 111.58 | 111.58 |
| | former | | | |
| Glycerol 85% | Plasticizer | 48.79 | 48.79 | 48.79 |
| Titanium | Colorant | 0.36 | 0.36 | 0.36 |
| dioxide | | | | |
| Iron oxide A | Colorant | 0.06 | 0.06 | 0.06 |
| Iron oxide B | Colorant | 0.17 | 0.17 | 0.17 |
| Total Capsule | | 440.96 | 440.96 | 440.96 |
| Weight | | | | |

### Example 6

Soft Gelatin Capsule Containing 125 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active Substance | Active | 150.50 | 150.50 | 150.50 |
| | Ingredient | | | |
| Triglycerides, | Carrier | 102.375 | 134.25 | 133.5 |
| Medium-chain | | | | |
| Hard fat | Thickener | 95.625 | 63.75 | 63.75 |
| Lecithin | Wetting | 1.50 | 1.50 | 2.25 |
| | agent/ | | | |
| | Glidant | | | |
| Gelatin | Film- | 142.82 | 142.82 | 142.82 |
| | former | | | |
| Glycerol 85% | Plasticizer | 62.45 | 62.45 | 62.45 |
| Titanium dioxide | Colorant | 0.47 | 0.47 | 0.47 |
| Iron oxide A | Colorant | 0.08 | 0.08 | 0.08 |
| Iron oxide B | Colorant | 0.22 | 0.22 | 0.22 |
| Total Capsule | | 556.04 | 556.04 | 556.04 |
| Weight | | | | |

### Example 7

Soft Gelatin Capsule Containing 150 mg of Active Substance

| | | Formulation | Formulation | Formulation |
|---|---|---|---|---|
| | | A | B | C |
| | | mg per | mg per | mg per |
| Ingredients | Function | capsule | capsule | capsule |
| Active Substance | Active | 180.60 | 180.60 | 180.60 |
| | Ingredient | | | |
| Triglycerides, | Carrier | 122.85 | 161.10 | 160.20 |
| Medium-chain | | | | |
| Hard fat | Thickener | 114.75 | 76.50 | 76.50 |
| Lecithin | Wetting | 1.80 | 1.80 | 2.70 |
| | agent/ | | | |
| | Glidant | | | |
| Gelatin | Film- | 142.82 | 142.82 | 142.82 |
| | former | | | |
| Glycerol 85% | Plasticizer | 62.45 | 62.45 | 62.45 |
| Titanium dioxide | Colorant | 0.47 | 0.47 | 0.47 |
| Iron oxide A | Colorant | 0.08 | 0.08 | 0.08 |
| Iron oxide B | Colorant | 0.22 | 0.22 | 0.22 |
| Total Capsule | | 626.04 | 626.04 | 626.04 |
| Weight | | | | |

### Example 8

Soft Gelatin Capsule Containing 200 mg of Active Substance

| | | Formulation | | Formulation |
|---|---|---|---|---|
| | | A | Formulation B | C |
| | | mg per | mg per | mg per |
| Ingredients | Function | capsule | capsule | capsule |
| Active | Active | 240.80 | 240.80 | 240.80 |
| Substance | Ingredient | | | |
| Triglycerides, | Carrier | 163.30 | 214.80 | 216.00 |
| Medium-chain | | | | |
| Hard fat | Thickener | 153.50 | 102.00 | 102.00 |
| Lecithin | Wetting | 2.40 | 2.40 | 1.20 |
| | agent/ | | | |
| | Glidant | | | |
| Gelatin | Film- | 203.19 | 203.19 | 203.19 |
| | former | | | |
| Glycerol 85% | Plasticizer | 102.61 | 102.61 | 102.61 |
| Titanium | Colorant | 0.57 | 0.57 | 0.57 |
| dioxide | | | | |
| Iron oxide A | Colorant | 0.90 | 0.90 | 0.90 |
| Iron oxide B | Colorant | 0.90 | 0.90 | 0.90 |
| Total Capsule | | 868.17 | 868.17 | 868.17 |
| Weight | | | | |

### Example 9

Bulk packaging materials for the packaging of the soft gelatin capsules of above examples 1 to 4 may be aluminium pouches or double poly bags.

### Example 10

In the following, a manufacturing process for the preparation of a lipid suspension formulation of the active substance and a process for the encapsulation are described.
a: Hard fat and parts of Medium-chain triglycerides are pre-mixed in the processing unit. Subsequently lecithin, the rest of medium-chain triglycerides and the active substance are added. The suspension is mixed, homogenized, de-aerated and finally sieved to produce the formulation (Fillmix).
b. The gelatin basic mass components are mixed and dissolved at elevated temperature. Then, the corresponding colours and additional water are added and mixed, producing the Coloured Gelatin Mass.
c. After adjustment of the encapsulation machine, Fillmix and Coloured Gelatin Mass are processed into soft gelatin capsules using the rotary-die process. This process is e.g. described in Swarbrick, Boylann, Encyclopedia of pharmaceutical technology, Marcel Dekker, 1990, Vol. 2, pp 269 ff.
d. After encapsulation, the traces of the lubricant medium-chain triglycerides are removed from the capsule surface, using ethanol denatured with acetone, containing small quantities of Phosal® 53 MCT, used here as anti-sticking agent.
e. The initial drying is carried out using a rotary dryer. For the final drying step, capsules are placed on trays. Drying is performed at 15−26° C. and low relative humidity.
f. After 100% visual inspection of the capsules for separation of deformed or leaking capsules, the capsules are size sorted and further washed using ethanol denatured with acetone.
g. Finally, the capsules are imprinted, using an Offset printing technology or an Ink-jet printing technology. Alternatively, the capsule imprint can be made using the Ribbon printing technology, a technology in which the gelatin bands are imprinted prior to the encapsulation step c.

Compound B1 (TAS-102, Lonsurf®) may be administered according to known clinical practice.

The dosages and schedules may vary according to the particular disease state and the overall condition of the patient. Dosages and schedules may also vary if, in addition to a combination treatment of the present invention, one or more additional chemotherapeutic agents is/are used. Scheduling can be determined by the practitioner who is treating any particular patient.

Radiotherapy may be administered according to the known practices in clinical radiotherapy. The dosages of ionising radiation will be those known for use in clinical radiotherapy. The radiation therapy used will include for example the use of γ-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0 Gy, 5 days a week for 5-6 weeks. Normally a total fractionated dose will lie in the range 45-60 Gy. Single larger doses, for example 5-10 Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

The size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient. For example, it may be necessary or desirable to reduce the above-mentioned doses of the components of the combination treatments in order to reduce toxicity.

1. A pharmaceutical combination comprising
(i) 3-Z-[1-(4-(N-((4-methyl-piperazin-1-yl)-methylcarbonyl)-N-methyl-amino)-anilino)-1-phenyl-methylene]-6-methoxycarbonyl-2-indolinone or a pharmaceutically acceptable salt thereof, and
(ii) 2′-deoxy-5-(trifluoromethyl)uridine or a pharmaceutically acceptable salt thereof, and
(iii) 5-chloro-6-[(2-iminopyrrolidin-1-yl)methyl]pyrimidine-2,4(1H,3H)-dione or a pharmaceutically acceptable salt thereof,
wherein the molar ratio of component (ii), 2′-deoxy-5-(trifluoromethyl)uridine or a pharmaceutically acceptable salt thereof, and component (iii), 5-chloro-6-[(2-iminopyrrolidin-1-yl)methyl]pyrimidine-2,4(1H,3H)-dione or a pharmaceutically acceptable salt thereof, is 1:0.5.

2. The pharmaceutical combination according to claim 1, in which the pharmaceutically acceptable salt of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone is its monoethanesulphonate salt form.

3. The pharmaceutical combination according to claim 1, in which the pharmaceutically acceptable salt of the compound 5-chloro-6-[(2-iminopyrrolidin-1-yl)methyl]pyrimidine-2,4(1H,3H)-dione is the hydrochloride form.

4. The pharmaceutical combination according to claim 1, comprising the monoethanesulphonate salt form of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone and the hydrochloride form of the compound 5-chloro-6-[(2-iminopyrrolidin-1-yl)methyl]pyrimidine-2,4(1H,3H)-dione.

5. The pharmaceutical combination according to claim 1, wherein component (ii) and (iii) form a mixture for simultaneous use of both components.

6. The pharmaceutical combination according claim 5, wherein component (i) and the mixture according to claim 5 is for simultaneous, separate or sequential use.

7.-30. (canceled)

31. A method of treating colorectal cancer or metastatic colorectal cancer, the method comprising administering the pharmaceutical combination according to claim 1 to a patient in need thereof.

32. The method according to claim 31, wherein the patient has been previously treated with a therapy selected from the group consisting of fluoropyrimidine-, oxaliplatin- and irinotecan-based chemotherapies, anti-VEGF agents, and anti-EGFR agents.

33. The method according to claim 31, wherein component (i) is administered in an amount of 100 mg of the free base, twice daily, 150 mg of the free base, twice daily, or 200 mg of the free base, twice daily.

34. The method according to claim 31, wherein component (ii) is administered in an amount of 35 mg of the free base per m², twice daily at days 1 to 5 and days 8 to 12 of a treatment cycle of 28 days and wherein component (iii) is to be administered simultaneously with component (ii).

35. The method according to claim 31, wherein the molar ratio of compound (ii), 2′-deoxy-5-(trifluoromethyl)uridine or a pharmaceutically acceptable salt thereof and compound (iii), 5-chloro-6-[(2-iminopyrrolidin-1-yl)methyl]pyrimidine-2,4(1H,3H)-dione or a pharmaceutically acceptable salt thereof, is 1:0.5.

36. The method according to claim 35, in which the pharmaceutically acceptable salt of the compound 3-Z-[1-(4-(N-((4-methyl-piperazin-1-yl)-methylcarbonyl)-N-methyl-amino)-anilino)-1-phenyl-methylene]-6-methoxycarbonyl-2-indolinone is its monoethanesulphonate salt form.

37. The method according to claim 36, in which the pharmaceutically acceptable salt of the compound 5-chloro-6-[(2-iminopyrrolidin-1-yl)methyl]pyrimidine-2,4(1H,3H)-dione is the hydrochloride form.

38. The method according to claim 36, wherein compounds (ii) and (iii) form a mixture for simultaneous use of both compounds.

39. The method according to claim 38, wherein compound (i) and the mixture of compounds (ii) and (iii) is for simultaneous, separate or sequential use.

40. The method according to claim 35, for use in a co-treatment with radiotherapy.

41. The method according to claim 31 for treatment of metastatic colorectal cancer.

42. The method according to claim 31 for treatment of colorectal cancer.

43. The method according to claim 31, where the patients were refractory or intolerant to standard therapies selected from the group consisting of fluoropyrimidine, irinotecan, oxaliplatin, anti-angiogenesis inhibitor and anti-EGFR antibody (if wild-type RAS).
